(19) United States
(12) Patent Application Publication
(10) Pub. No.: US 2018/0303955 A1
(43) Pub. Date: Oct. 25, 2018

(54) METHODS AND RELATED COMPOSITIONS FOR THE TREATMENT OF CANCER

(71) Applicant: IMMIX BIOPHARMA, INC, Los Angeles, CA (US)

(72) Inventor: Ilya Rachman, Los Angeles, CA (US)

(73) Assignee: IMMIX BIOPHARMA, INC, Los Angeles, CA (US)

(21) Appl. No.: 15/769,586

(22) PCT Filed: Oct. 21, 2016
(86) PCT No.: PCT/US2016/058159

§ 371 Date: Apr. 19, 2018

Related U.S. Application Data
(60) Provisional application No. 62/245,813, filed on Oct. 23, 2015.

(51) Int. Cl. A61K 47/69; A61K 47/68; A61K 31/12; A61K 31/704; A61K 9/00; A61P 35/00

(52) U.S. Cl. CPC A61K 47/6909; A61K 47/6849; A61K 31/12; A61P 35/00; A61K 9/0019; A61K 47/6803; A61K 31/704

(57) ABSTRACT

Disclosed herein are compositions and nanoconjugates comprising a micelle construct; an antibody single chain fragment variable (scFv); a NF-kb inhibitor; and a topoisomerase II inhibitor. Further disclosed herein are methods of delivering a drug molecule to a tumor site of a subject comprising: attaching the drug molecule to a targeted micelle, wherein the targeted micelle comprises a micelle construct and an antibody single chain fragment variable (scFv); and delivering the drug molecule attached to the targeted micelle to the tumor site through intracellular delivery. Also disclosed herein are methods of treating cancer or inhibiting tumor cell growth comprising administering to a subject in need thereof, a therapeutically effective dosage of a composition comprising a micelle construct, an antibody single chain fragment variable (say), a NF-kb inhibitor, and a topoisomerase II inhibitor.

### RELATED APPLICATION

This application claims the benefit of priority of U.S. Provisional Patent Application No. 62/245,813, filed on Oct. 23, 2015, which is incorporated by reference herein in its entirety,

### FIELD OF THE INVENTION

The present disclosure relates to the field of medicine, and more specifically, the treatment of cancer.

### BACKGROUND OF THE DISCLOSURE

Complete and effective treatment for cancer has not been developed despite billions of dollars being spent in cancer research. Part of the reason is because tumor cells can be made up of a variety of cell types, produced as the cells proliferate and incur different mutations. This diversity, in turn, is part of what has made treatment of cancer so difficult, as a population of cancerous cells could easily include a mutant variety that happens to be resistant to any individual treatment or chemotherapy drug that is administered. The few resistant cancer cells are provided a strong selective advantage in comparison to other cells, and over time, those resistant cells increase in frequency.

Thus, there is a need in the art for the development of additional cancer treatments, including those that have the ability to better target drug resistant tumors and potentially bypass the diversity of cancer cells.

### SUMMARY OF THE INVENTION

In one embodiment, disclosed herein are compositions, comprising, a micelle construct; an antibody single chain fragment variable (scFv); a NE-kb inhibitor; and a topoisomerase H inhibitor. In one embodiment, the say is conjugated to the micelle construct. In one embodiment, the micelle construct is conjugated to the NE-kb inhibitor. In one embodiment, the micelle construct is conjugated to the NE-kb inhibitor and the topoisomerase II inhibitor. In one embodiment, the NE-kb inhibitor is curcumin or a pharmaceutical equivalent, analog, derivative, and/or salt thereof In one embodiment, the topoisomerase II inhibitor is dox or a pharmaceutical equivalent, analog, derivative, and/or salt thereof. In one embodiment, the say is a glut-1_1 or glut-1_2 scFv. in one embodiment, the micelle construct is targeted to bind to glut-1_1 and/or glut-1_2 scFv. In one embodiment, the composition forms a targeted micelle. In one embodiment, the molecular size of the targeted micelle is 30 nm or less. In one embodiment, the composition further comprises a pharmaceutically acceptable carrier.

In another embodiment, disclosed herein are methods of delivering a drug molecule to a tumor site of a subject comprising: attaching the drug molecule to a targeted micelle, wherein the targeted micelle comprises a micelle construct and an antibody single chain fragment variable (scFv); and delivering the drug molecule attached to the targeted micelle to the tumor site through intracellular delivery. In one embodiment, the drug molecule comprises dox and/or curcumin or a pharmaceutical equivalent, analog, derivative, and/or salt thereof in one embodiment, the molecular size of the micelle is 30 nm or less. In one embodiment, the tumor is a doxorubicin-resistant tumor. In one embodiment, the subject is human.

In another embodiment, disclosed herein are methods of treating cancer comprising administering to a subject in need thereof, a therapeutically effective dosage of a composition comprising a micelle construct, an antibody single chain fragment variable (scFv), a NF-kb inhibitor, and a. topoisomerase II inhibitor. In one embodiment, the composition is administered to the subject intravenously. In one embodiment, the subject is human. In one embodiment, the cancer is brain cancer. In one embodiment, the cancer is ovarian cancer. In one embodiment, the cancer is colon cancer. In one embodiment, the cancer is a doxorubicin-resistant cancer. In one embodiment, the molecular size of the targeted micelle is 30 nm or less.

In another embodiment, disclosed herein are methods of inhibiting cell growth of a tumor cell, comprising: providing a composition comprising a micelle construct, an antibody single chain fragment variable (scFv), a NF-kb inhibitor and a topoisomerase II inhibitor; and inhibiting cell growth by administering a therapeutically effective dosage of the composition to the tumor cell. In one embodiment, the topoisomerase inhibitor comprises Dox. In one embodiment, the NF-kB inhibitor comprises curcumin. In one embodiment, the tumor cell is a brain cancer cell, a ovarian cancer cell, and/or colon cancer cell.

In another embodiment, disclosed herein are nanoconjugates comprising: a micelle construct; an antibody single chain fragment variable (scFv); a NF-kb inhibitor; and a topoisomerase II inhibitor; and wherein the micelle construct, the antibody single chain fragment variable (scFv), the NF-kb inhibitor, and the topoisomerase II inhibitor are conjugated to one another. In one embodiment, the nanoconjugate has a molecular size between 20 nm and 50 nm. In one embodiment, the nanoconjugate has a molecular size between 10 nm and 20 nm. In one embodiment, the nanoconjugate has a molecular size of less than 20 nm. In one embodiment, the nanoconjugate is enclosed by a micelle.

### DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with the embodiments herein, the efficacy of CUR40/DOX0.8 micelles on Doxorubicin-resistant ovarian carcinoma cell lines, A2780/Adr, after 48 hours of continuous contact. The columns show the cell viability at CUR/DOX concentrations 80/1.6, 40/0.8, 20/0.4, 10/0.2, 5/0.1, 2.5/0.05, 1.25/0.025. Experimental data compares the cell viability of EmptyMicelles, G1_-EmptyMicelles, G1_2-EmptyMicelles, CUR40/DOX0.8 Micelles, G1_1-CUR40/DOX0.8 Micelles, and G1_2-CUR40/DOX0.8 micelles.

FIG. 2 depicts, in accordance with the embodiments herein, the efficacy of free CUR, free DOX, and free CUR/DOX on Doxorubicin-sensitive ovarian carcinoma cell lines, A2780/Sens, after 4-44 hours of contact. Experimental data compares the cell viability of free CUR, free DOX, and free CUR/DOX at CUR/DOX (uM) concentrations: 40/0.8, 20/0.4, 10/0.2, 5/0.1, 2.5/0.05, and 1.25/0.025.

FIG. 3 depicts, in accordance with the embodiments herein, the efficacy of Empty and CUR-only micelles on Doxorubicin-sensitive ovarian carcinoma cell lines, A2780/Sens, after 4-44 hours of contact. The data compares the cell viability (%) of Empty Micelles, G1_1-EmptyMicelles, G1_2-EmptyMicelles, CUR Micelles, CUR-G1_1 Micelles and CUR-G1_2 Micelles at CUR/DOX (uM) concentrations 40/0.8, 20/0.4, 10/0.2, 5/0.1, 2.5/0.05, and 1.25/0.025.

FIG. 4 depicts, in accordance with the embodiments herein, the efficacy of CUR and CUR/DOX micelles on Doxorubicin-sensitive ovarian carcinoma cell lines, A2780/Sens, after 4-44 hours of contact. The columns show the cell viability at CUR/DOX concentrations 40/0.8, 20/0.4, 10/0.2, 5/0.1, 2.5/0.05, and 1.25/0.025. Experimental data compares the cell viability of CUR Micelles, CUR-G1_1 Micelles, CUR-G1 Micelles, CUR/DOX Micelles, CUR/DOX-G1_1 Micelles, and CUR/DOX-G1_2 Micelles.

FIG. 5 depicts, in accordance with the embodiments herein, the efficacy of empty and CUR-only micelles on Doxorubicin-resistant ovarian carcinoma cell lines, A2780/Adr after 4-44 hours of contact. The columns show the cell viability at CUR/DOX (uM) concentrations 40/0.8, 20/0.4, 10/0.2, 5/0.1, 2,5/0.05, and 1.25/0.025. Experimental data compares the cell viability of Empty Micelles, G1_1-Empty MiCelles, G1_2-Empty MiCelles, CUR Micelles, CUR-G1_1 Micelles, and CUR-G1_2 Micelles.

FIG. 6 depicts, in accordance with the embodiments herein, the efficacy of CUR and CUR/DOX micelles on Doxorubicin-resistant ovarian carcinoma cell lines, A2780/Adr after 4-44 hours of contact. The columns show the cell viability at CUR/DOX (uM) concentrations 40/0.8, 20/0.4, 10/0.2, 5/0.1, 2.5/0.05, and 1.25/0.025. Experimental data compares the cell viability of CUR Micelles, CUR-G1_1 Micelles, CUR-G1_2 Micelles, CUR/DOX Micelles, CUR/DOX-G1_1 Micelles, and CUR/DOX-G1_2 Micelles.

FIG. 7 depicts, in accordance with the embodiments herein, the efficacy of CUR40/DOX0.8 micelles on Doxorubicin-sensitive ovarian carcinoma cell lines. A2780/Sen after 24 hours of continuous contact, The columns show the cell viability at CUR/DOX (uM) concentrations 80/1.6, 40/0.8, 20/0.4, 10/0.2, 5/0.1, 2.5/0.05, 1.25/0.025, and 0.625/0.0125. Experimental data compares the cell viability of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles.

FIG. 8 depicts, in accordance with the embodiments herein, the efficacy of CUR40/DOX0.8 micelles on Doxorubicin-sensitive ovarian carcinoma cell lines, A2780/Sen, after 48 hours of continuous contact. The columns show the cell viability at CUR/DOX (uM) concentrations 80/1.6, 40/0.8, 20/0.4, 10/0.2, 5/0.1, 2.5/0.05, and 1.25/0.025. Experimental data compares the cell viability of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles.

FIG. 9 depicts, in accordance with the embodiments herein, the efficacy of CUR40/DOX0.8 micelles on Doxorubicin-resistant ovarian carcinoma cell lines, A2780/Adr after 24 hours of continuous contact. The columns show the cell viability at CUR/DOX (uM) concentrations 80/1.6, 40/0.8, 20/0.4, 10/0.2, 5/0.1, 2.5/0.05, and 1.25/0.025. Experimental data compares the cell viability of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles.

FIG. 10 depicts, in accordance with the embodiments herein, the efficacy of CUR40/DOX0.8 micelles on Doxorubicin-resistant ovarian carcinoma cell lines, A2780/Adr, after 48 hours of continuous contact. The columns show the cell viability at CUR/DOX (uM) concentrations 80/1,6, 40/0.8, 20/0.4, 10/0.2, 5/0.1, 2.5/0.05, and 1.25/0.025. Experimental data compares the cell viability of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles.

FIG. 11 depicts, in accordance with the embodiments herein, the efficacy of free CUR, free DOX, and free CUR/DOX on HCT-116 cells after 4-44 hours of contact. The columns illustrate the cell viability (%) at CUR/DOX (uM) concentrations: 40/0.8, 20/0.4, 10/0.2, 5/0.1, 2.5/0.05, and 1.25/0.025.

FIG. 12 depicts, in accordance with the embodiments herein, the efficacy of CUR and CUR/DOX micelles on HCT-116 cells after 4-44 hours of contact. The columns show the cell viability at CUR/DOX (uM) concentrations 40/0.8, 20/0.4, 10/0.2, 5/0.1, 2.5/0.05, and 1.25/0.025. Experimental data compares the cell viability of CUR Micelles, CUR-G1_1 Micelles, CUR-G1_2 Micelles, CUR/DOX Micelles, CUR/DOX-G1_1 Micelles, and CUR/DOX-G_2 Micelles.

FIG. 13 depicts, in accordance with the embodiments herein, the efficacy of CUR40/DOX0.8 micelles HCT-116 cells after 24 hours of continuous contact. The columns show the cell viability at CUR/DOX (uM) concentrations 80/1.6, 40/0.8, 20/0.4, 10/0.2, 5/0.1, 2.5/0.05, and 1.25/0.025. Experimental data compares the cell viability (%) of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles.

FIG. 14 depicts, in accordance with the embodiments herein, the efficacy of CUR40/DOX0.8 micelles on HCT-116 cells after 48 hours of continuous contact. The columns show the cell viability at CUR/DOX (uM) concentrations 80/1.6, 40/0.8, 20/0.4, 10/0.2, 5/0.1, 2.5/0.05, and 1.25/0.025. Experimental data compares the cell viability (%) of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles.

FIG. 15 depicts, in accordance with the embodiments herein, the efficacy of free drugs and micelles on U87MG cells after 24 hours of continuous contact. The columns show the cell viability at CUR/DOX (uM) concentrations 40/0.8, 20/0.4, 10/0.2, 5/0.1, 2.5/0.05, and 1.25/0.025. Experimental data compares the cell viability (%) of free CUR, free DOX, free CUR/DOX, CUR Micelles, CUR-G1_1 Micelles, CUR-G1_2 Micelles, CUR/DOX Micelles, CUR/DOX-G1_1 Micelles, and CUR/DOX-G1_2 Micelles.

FIG. 16 depicts, in accordance with the embodiments herein, the efficacy of free drugs and micelles on U87MG cells after 48 hours of continuous contact. The columns show the cell viability (%) at CUR/DOX (uM) concentrations 40/0.8, 20/0.4, 10/0.2, 5/0.1, 2.5/0.05, and 1.25/0.025. Experimental data compares the cell viability of free CUR, free DOX, free CUR/DOX, CUR Micelles, CUR-G1_1 Micelles, CUR-G1_2 Micelles, CUR/DOX Micelles, CUR/DOX-G1_1 Micelles, and CUR/DOX-G1_2 Micelles.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and Materials similar or equivalent to those described herein, which could be used in the practice of the present invention. Indeed, the present invention is in no way limited to the methods and materials described.

As used herein, “HCT-116” refers to a colon tumor cell line. As used herein, “A2780” refers to an ovarian cancer cell line. As used herein, “U87MG” or “U87” refers to a brain cancer cell line.

As used herein, the abbreviation of “CUR” refers to curcumin. As used herein, the term “DOX” refers to doxorubicin.

As used herein, the terms “glut-1,” “GLUT-1,” “G1,” or “glut1,” used interchangeably, refers to a glucose transporter antibody. The term scFv, as used herein, refers to single chain variable fragment. Thus, the terms GLUT-1_1 and/or GLUT1_2 scFv refers to a single chain variable fragment of the GLUT-1 antibody.

As described herein, the inventor has recognized that effective cancer treatment would benefit from attacking the cancer early, as well as attacking aggressively. This could come in the form of administering a combination of drugs for treatment, as the odds of a single cell being resistant to a larger quantity of drugs are lower. Additionally, an effective cancer treatment could also potentially bypass the diversity of cancer cells by targeting processes that cancer cells rely on for their very growth. One such process is tumors' reliance on producing and processing sugar for its cell growth.

As described herein, the inventor has developed various compositions and methods for the treatment of cancers and associated conditions. In accordance with embodiments further described herein, the inventor developed and optimized cancer therapeutic compositions and methods for effective delivery and minimized toxicity. For example, in one embodiment, utilizing a single chain antibody allows a more efficient delivery in conjunction with various embodiments herein. In accordance with various embodiments further disclosed herein, the inventor also attached curcumin to a targeted micelle. By attaching curcumin to a targeted micelle, treatment was administered at a significantly lower dosage, thus reducing toxicity while effectively inhibiting tumor growth. As compared to the liposomal forms of both doxorubicin and curcumin, a micellar preparation is of significantly smaller molecular size (10-20 nm vs. 80-150 nm liposomes) resulting in improved tumor mass penetration from the vascular bed, thus creating a more effective cancer therapeutic.

In one embodiment, disclosed herein are compositions comprising a micelle construct; an antibody single chain fragment variable (scFv); a NF-kb inhibitor; and a topoisomerase II inhibitor. In one embodiment, the scFv is conjugated to the micelle construct In one embodiment, the micelle construct is conjugated to the NF-kb inhibitor. In one embodiment, the micelle construct is conjugated to the NF-kb inhibitor and the topoisomerase II inhibitor. In one embodiment, the NF-kb inhibitor is curcumin or a pharmaceutical equivalent, analog, derivative, and/or salt thereof. In one embodiment, the topoisomerase II inhibitor is dox or a pharmaceutical equivalent, analog, derivative, and/or salt thereof. In one embodiment, the scFv is a glut-1_1 or glut-1_2 scFv. In one embodiment, the micelle construct is targeted to bind to glut-1_1 and/or glut-1_2 scFv. In one embodiment, the composition forms a targeted micelle. In one embodiment, the molecular size of the targeted micelle is 30 nm or less. In one embodiment, the composition further comprises a pharmaceutically acceptable carrier.

In another embodiment, disclosed herein are methods of delivering a drug molecule to a tumor site of a subject comprising: attaching the drug molecule to a targeted micelle, wherein the targeted micelle comprises a micelle construct and an antibody single chain fragment variable (scFv); and delivering the drug molecule attached to the targeted micelle to the tumor site through intracellular delivery. In one embodiment, the drug molecule comprises dox and/or curcumin or a pharmaceutical equivalent, analog, derivative, and/or salt thereof. In one embodiment, the molecular size of the micelle is 30 nm or less. In one embodiment, the tumor is a doxorubicin-resistant tumor. In one embodiment, the subject is human.

In another embodiment, disclosed herein are methods of treating cancer comprising administering to a subject in need thereof, a therapeutically effective dosage of a composition comprising a micelle construct, an antibody single chain fragment variable (scFv), a NF-kb inhibitor, and a topoisomerase II inhibitor. In one embodiment, the composition is administered to the subject intravenously. In one embodiment, the subject is human. In one embodiment, the cancer is brain cancer. In one embodiment, the cancer is ovarian cancer. In One embodiment, the cancer is colon cancer. In one embodiment, the cancer is a doxorubicin-resistant cancer. In one embodiment, the molecular size of the targeted micelle is 30 nm or less.

In another embodiment, disclosed herein are methods of inhibiting cell growth of a tumor cell, comprising: providing a composition comprising a micelle construct, an antibody single chain fragment variable (scFv), a NF-kb inhibitor and a topoisomerase II inhibitor; and inhibiting cell growth by administering a therapeutically effective dosage of the composition to the tumor cell. In one embodiment, the topoisomerase inhibitor comprises Dox. In one embodiment, the NF-kB inhibitor comprises curcumin. In one embodiment, the tumor cell is a brain cancer cell, a ovarian cancer cell, and/or colon cancer cell.

In another embodiment, disclosed herein are nanoconjugates comprising: a micelle construct; an antibody single chain fragment variable (scFv); a NF-kb inhibitor; and a topoisomerase II inhibitor; and wherein the micelle construct, the antibody single chain fragment variable (scFv), the NF-kb inhibitor, and the topoisomerase II inhibitor are conjugated to one another. In one embodiment, the nanoconjugate is between 20 nm and 50 nm. In one embodiment, the nanoconjugate is between 10 nm and 20 nm. In one embodiment, the nanoconjugate is less than 20 nm. In one embodiment, the nanoconjugate is enclosed by a micelle.

Embodiments of the present disclosure are further described in the following examples. The examples are merely illustrative and do not in any way limit the scope of the invention as claimed.

### EXAMPLES

### Example 1

### Materials

1,2-Distearoyl-sn-glycero-3-phosphoethanolamine-N-[methoxy (polyethylene glycol)-2000] (PEG-PE) was from Avanti Polar Lipids (Alabaster, Ala., USA). pNP-PEG₃₄₀₀-pNP was purchased from Laysan Bio (Arab, Ala.). Curcumin (CUR), vitamin E and triethylamine (TEA) were purchased from Sigma (St. Louis, Mo., USA). Doxorubicin HCl (DOX) was from LC Laboratories (Woburn, Mass.). Accumax was from Innovative Cell Technologies, Inc. (San Diego, Calif.). The CellTiter-Glo luminescent cell viability assay kit was from Promega (Fitchburg, Wis.). All other reagents were of analytical grade. DOX free base was obtained by incubating DOX HCl (in methanol, 0.5 mg/ml) with 5-fold molar excess of TEA overnight. pNP-PEG₃₄₀₀-PE was synthesized in-house.

### Example 2

### Micelle Preparation

DOX and/or CUR drug-loaded mixed micelles were prepared by the thin film hydration method. CUR (1 mg/ml in 0.1% acetic acid-methanol solution) and/or DOX free base (0.5 mg/ml in methanol solution) were added to PEG₂₀₀₀-PE solution in chloroform. Initial drug amounts were adjusted after the formulation optimization studies to result in CUR:DOX ratio of 32 (w/w). The organic solvents were removed by the rotary evaporation and a thin film of drugs/micelle-forming material mixture was formed. This film was further dried overnight in freeze-dryer to remove any residuals of organic solvents (Freezone 4.5 Freeze Dry System, Labconco, Kansas City, Mo.). Drug-loaded micelles were formed by resuspending the film in phosphate buffered saline (PBS) pH 7.2, to give the final concentration of micelle forming materials of 5 mM in all formulations. The micelle formulations were dialyzed 1 h against PBS pH 7.2 to remove excess of TEA. Excess non-incorporated drugs were separated by filtration through a 0.2 μm syringe filter.

To obtain targeted micelles, GLUT-1 scFv was reacted with pNP-PEG₃₄₀₀-PE. Briefly, required amount of pNP-PEG₃₄₀₀-PE in chloroform was added into the round bottom flask and polymer film was formed after removing the solvent by rotary evaporation followed bye drying under vacuum. The film was hydrated and vortexed first with citrate-buffered saline (CBS) pH 5.0 to prevent early hydrolysis of pNP distal groups. After forming micelles, GLUT-1 scFv solution in PBS (pH 7.4) was added to the mixture, and the pH was adjusted to 8.2 with NaOH. Molar ratio of pNP groups to scFv was kept at 40:1. Reaction time was overnight at 4° C. to allow sufficient conjugation and the complete hydrolysis of the unreacted pNP groups at the higher pH. Targeted-micelles were then dialyzed using a 50,000 MWCO cellulose ester membrane against PBS (pH 7.4) for 4 hours at 4° C. to ensure the complete removal of the unconjugated scFv. Conjugation efficiency of GLUT-1 scFv was measured using a micro BCA kit (Pierce, Rockford, Ill.) according to the manufacturer's instructions. GLUT-1 scFv conjugated micelles were mixed and co-incubated with drug loaded PEG-PE micelles for 4 hours at room temperature. The final GLUT-1 scFv mol ratio in the micelles was adjusted to 0.05%.

### Example 3

### GLUT-1 scFv Modified Micelle Cytotoxicity on Ovarian Cancer Cell Line

For cytotoxicity evaluations, A2780 human ovarian carcinoma (ATCC) and its Doxorubicin resistant derivative A2780/Adr (ECACC) were used. A2780 cells were cultured in RPMI medium supplemented with 10% FBS and 1% penicillin-streptomycin. Drug resistant ovarian cancer cells were grown in same medium with the addition of 100 nM DOX.

The cells were seeded in 96-well plates at a density of 3000 cells/well or 5,000 cells/well for 48 or 24 hours treatment groups, respectively. The cells were treated with free drugs or micelle formulations containing 40 μM CUR and 0.8 μM DOX in serum complete RPMI medium. The cells were incubated 24 h and 48 h continuously in the continuous treatment groups. Additionally, in another treatment regimen the cells were incubated with drugs/micelles for 4 hours then washed and further incubated for 44 h. At the end of treatment times, the wells were washed twice with medium and then incubated with 50 μl medium containing 20% (v/v) CellTiter-Blue reagent. The fluorescent emission values were measured and % cell viability was calculated by using PBS treated cells as the control group.

### Example 4

### 3D Glioblastoma Spheroids Preparation and Cytotoxicity Evaluation of Micelles

The U87MG cells were cultured in DMEM medium supplemented with 10% FBS and 1% penicillin-streptomycin. For monolayer cytotoxicity experiments 3000 or 5000 cells were seeded into the each well of 96-well plate for 48 h and 24 h continuous treatment times, respectively. After overnight incubation of the plates, the cells were treated with CUR and/or DOX as free drugs or micellar formulations. CellTiter-Blue was used as the cytotoxicity evaluation method as described above.

For 3D cancer cell spheroid preparation, liquid overlay method was used. Briefly, serum free DMEM medium with 1.5% (w/v) agar was prepared and sterilized. 50 μL of the agar solution were added to the bottom of each well of the 96 well plates to prevent cell adhesion onto the well surface. Plates were allowed to cool down for 45 minutes before use. U87MG cells were trypsinized, counted and then seeded at the density of 10,000 cells/well. Plates were centrifuged for 15 min at 1,500 ref at 24° C. Spheroid formation was continuously followed using Nikon Eclipse E400 microscope (Nikon Inc., Melville, N.Y.) with the Spot Insight camera and Spot Advanced software (Spot imaging, Sterling Heights, Mich.). When the spheroids are formed and dense after 5 days, they were treated with the formulations. Cellular viability of the spheroids was determined after 48 h treatment. After the treatment with different formulations, drug concentrations and combinations, all the media from the corresponding wells along with the spheroids were collected and placed in a centrifuge tube. Five spheroids were collected as one replicate to increase the sensitivity. The spheroids were washed two times with PBS. Following removal of the remaining PBS, AccuMax® cell detachment solution was added and tubes were incubated for 10 minutes at 37° C. on the horizontal shaker with occasional pipetting. After dispersing of the spheroids into single cells, FBS was added into the tubes. Obtained cells were centrifuged and supernatants were discarded. Complete DMEM and CellTiter-Glo reagent at 1:1 volume ratio was added into the tubes and cells were incubated for 20 minutes for complete cell lysis. Luminescence was measured using multiplate reader by transferring 100 μL of the solution into 96 well black-walled plates.

### Example 5

### Compositions and Methods

As described herein, the inventor has developed various compositions and methods for the treatment of cancers and associated conditions. In one embodiment, the present invention provides a method of treating cancer and/or inhibiting growth in a tumor cell in a subject, by providing a composition comprising a micelle targeted by a glut-1 receptor antibody and attached to one or more curcumin and/or dox molecules, and administering a therapeutically effective dosage to the subject. In another embodiment, the composition is administered to the subject intravenously. In another embodiment the glut-1 receptor antibody is a single chain antibody. In another embodiment, the glut-1 receptor antibody is GLUT-1_1 and/or GLUT1_2 scFV. In another embodiment, the tumor cell is an ovarian tumor. In another embodiment, the tumor cell is a brain tumor. In another embodiment, the tumor cell is A2780. In another embodiment, the tumor cell is U87MG.

In accordance with embodiments further described herein, the inventor developed and optimized cancer therapeutic compositions and methods for effective delivery and minimized toxicity. For example, in one embodiment, utilizing a single chain antibody allowed a more efficient delivery in conjunction with various embodiments herein. Also, for example, by utilizing dox attached to a targeted micelle as a lipid-based delivery vehicle, rather than liposomal dox, or just dox, for example, the inventor created a cancer treatment with significantly high penetration of tumor mass. in addition to creating high tumor mass penetration, administering a composition comprising a dox attached to a targeted micelle optimized intracellular delivery of dox within the tumor cell itself Because dox acts as a weakly basic compound, if it enters a low pH environment or the lysosome of a tumor cell for example, the dox can lose much of its effectiveness for inhibiting tumor cell growth. By administering dox attached to a targeted micelle, rather than administering liposomal dox for example, the inventor enabled the dox to instead enter the cytoplasm, thus optimizing intracellular delivery. Additionally, dox can have high toxicity which can thus limit its practical application in vivo and usefulness as a cancer treatment for human subjects. In contrast, the inventor administered dox attached to a targeted micelle, resulting in further optimization of its effectiveness as a cancer therapeutic.

In accordance with various embodiments further disclosed herein, the inventor also attached curcumin to a targeted micelle. Due to its non-soluble properties, if administered directly, curcumin must be administered at high concentrations to be effective inhibiting tumor growth. However, at those same high concentrations, curcumin results in high toxicity, thus making it an impractical and ineffective cancer treatment in vivo, and particularly difficult for use in human patients. In accordance with an embodiment herein, by attaching curcumin to a targeted micelle, treatment can be administered at a significantly lower dosage, thus reducing toxicity while effectively inhibiting tumor growth.

As compared to the liposomal forms of both doxorubicin and curcumin, a micellar preparation is of significantly smaller molecular size (10-20 nm vs. 80-150 nm liposomes) resulting in improved tumor mass penetration from the vascular bed, thus creating a more effective cancer therapeutic. Additionally, in accordance with an embodiment herein, the addition of the Glut-1 Ab to the micelle greatly increased its therapeutic efficacy over the un-targeted micelle preparations through improved intracellular delivery of its contents. Glut-1 presents an attractive extracellular target since it is one of the main glucose transporters involved in tumor glucose uptake. Solid tumors can take up glucose at much higher rates than do normal cells. Glycolysis represents a main source of energy and carbon building blocks for growing tumors. Thus, in accordance with an embodiment herein, a micelle construct with a Glut-1 Ab is an effective cancer therapeutic, as glut-1 overexpression will persist even in the face of tumor phenotypic evolution, and it will be difficult for tumors to mutate away from glut-1 overexpression and still retain their high growth potential. In regard to using glut-1 as the tumor targeting entity, by binding to the tumor membrane-overexpressed glut-1, various embodiments of therapeutic micelles described herein get endocytosed into the cytoplasm rather than the low-pH lysosome, thereby increasing the therapeutic efficacy of doxorubicin (which has much lower activity at low pH).

Since the tumor lines used in these experiments were doxorubicin-resistant, it was critical that curcumin delivery occurred contemporaneously with doxorubicin exposure in Order for it to exert its tumoricidal effect. As NFκB overexpression and its attendant apoptosis- and chemotherapy-resistance are present in advanced cancers, the inventor designed various embodiments herein to include an NFκB inhibitor (for example, curcumin) in order to unlock the cidal effect of doxorubicin. In one embodiment, the present invention is a tumor-targeted micelle containing a tumor-tidal agent coupled with an apoptosis inhibitor with significant in vivo tumor inhibitory effect and clear applicability to human cancer therapeutics.

### Example 6

### Efficacy on Chemo-Resistant Ovarian Carcinoma Cells (A2780/Adr)

In one embodiment, the inventor has demonstrated the efficacy of the compounds and micelles disclosed herein in studies on Doxorubicin-resistant ovarian carcinoma cells. At 20/0.4 uM CUR/DOX concentration, the enhancing effect of the Glut1 antibody targeting is clearly visible with reduced tumor cell viability.

As illustrated in FIG. 1, the efficacy of CUR40/DOX0.8 micelles on Doxorubicin-resistant ovarian carcinoma cell lines, A2780/Adr, after 48 hours of continuous contact has been determined. The columns show the cell viability at CUR/DOX concentrations 80/1.6, 40/0.8, 20/0.4, 10/0.2, 5/0.1, 2.5/0.05, 1.25/0.025. Experimental data comparing the cell viability of EmptyMicelles, G1_1-EmptyMicelles, G1_2-EmptyMicelles, CUR40/DOX0.8 Micelles, G1_1-CUR40/DOX0.8 Micelles, and G1_2-CUR40/DOX.0.8 micelles is shown.

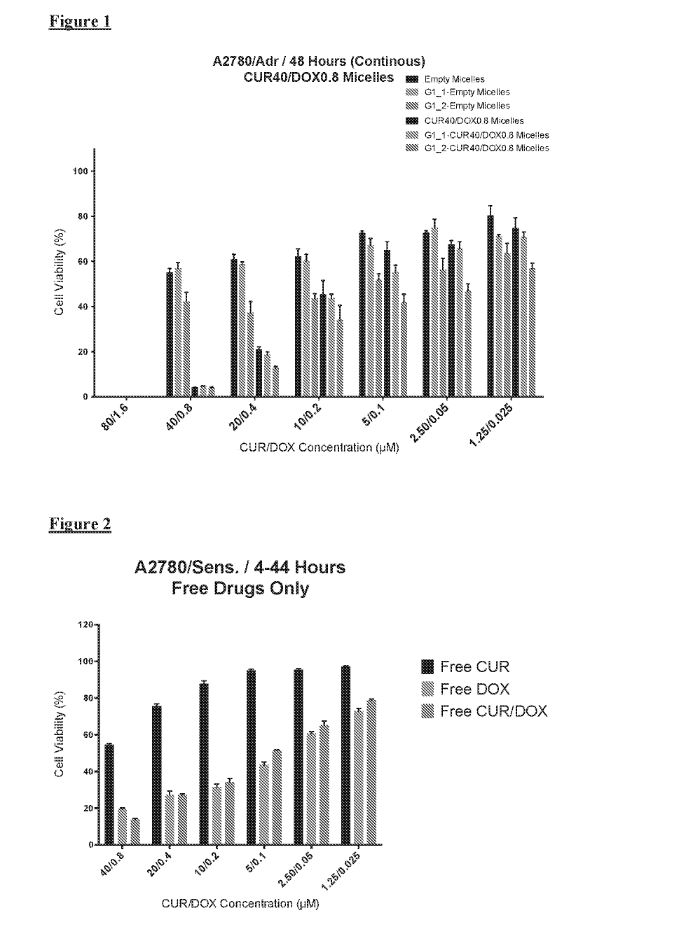

As illustrated in FIG. 2, the efficacy of free CUR, free DOX, and free CUR/DOX on Doxorubicin-sensitive ovarian carcinoma cell lines. 42780/Sens, after 4-44 hours of contact has been determined. Experimental data comparing the cell viability of free CUR, free DOX, and free CUR/DOX at CUR/DOX (uM) concentrations: 40/0.8, 20/0.4, 10/0.2, 5/0.1, 2.5/0.05, and 1.25/0.025 is shown.

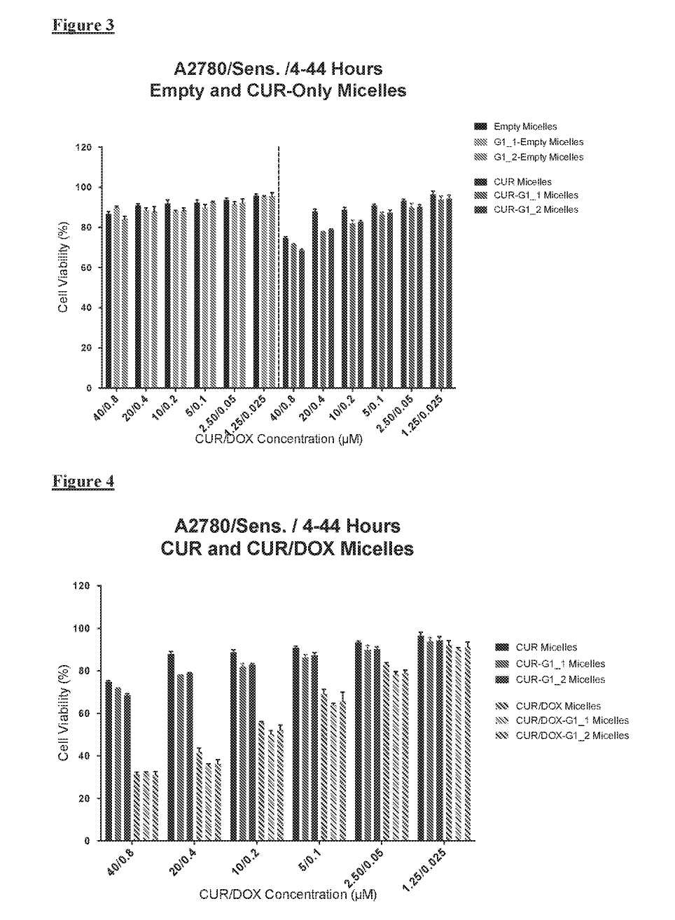

As illustrated in FIG. 3, the efficacy of empty and CUR-only micelles on Doxorubicin-sensitive ovarian carcinoma cell lines, A2780/SenS, after 4-44 hours of contact has been determined. The data comparing the cell viability (%) of EmptyMicelles, G1_1-EmptyMicelles, G1_2-EmptyMicelles, CUR Micelles, CUR-G1_1 Micelles and CUR-G1_2 Micelles at CUR/DOX (uM) concentrations 40/0.8, 20/0,4, 10/0.2, 5/0.1, 2.5/0.05, and 1.25/0.025 is shown.

As illustrated in FIG. 4, the efficacy of CUR and CUR/DOX micelles on Doxorubicin-sensitive ovarian carcinoma cell lines, A2780/Sens, after 4-44 hours of contact has been determined. The columns show the cell viability at CUR/DOX concentrations 40/0.8, 20/0.4, 10/0.2, 5/0.1, 2.5/0.05, and 1.25/0.025. Experimental data comparing the cell viability of CUR Micelles, CUR-G1_1 Micelles, CUR-G1 Micelles, CUR/DOX Micelles, CUR/DOX-G1_1 Micelles, and CUR/DOX-G1_2 Micelles is shown.

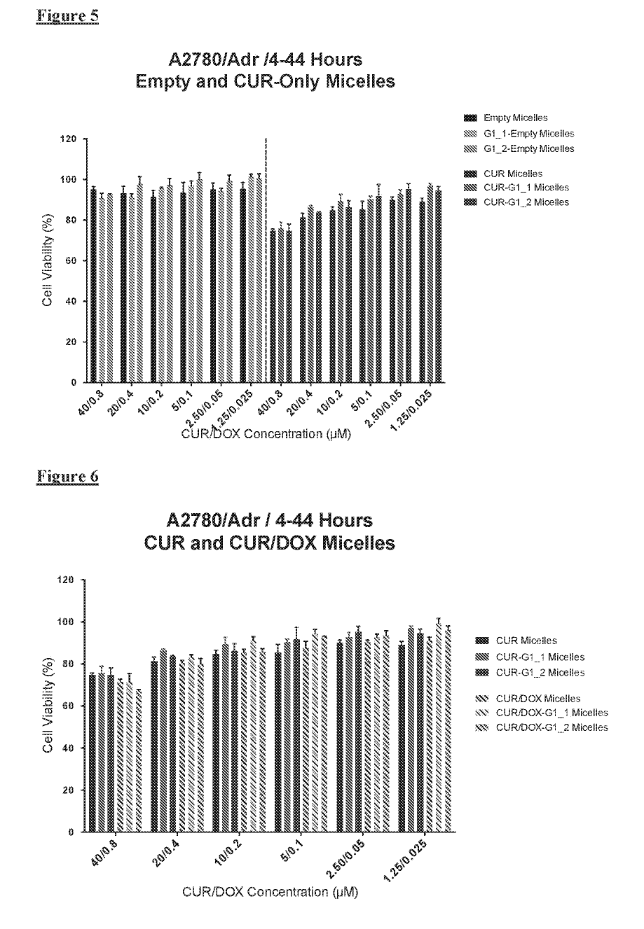

As illustrated in FIG. 5, the efficacy of empty and CUR-only micelles on Doxorubicin-resistant ovarian carcinoma cell lines, A2780/Adr after 4-44 hours of contact has been determined. The columns show the cell viability at CUR/DOX (uM) concentrations 40/0.8, 20/0.4, 10/0.2, 5/0.1, 2.5/0.05, and 1.25/0.025. Experimental data comparing the cell viability of Empty Micelles, G1_1-Empty Micelles, G1_2-Empty Micelles, CUR Micelles, CUR-G1_1 Micelles, and CUR-G1_2 Micelles is shown.

As illustrated in FIG. 6, the efficacy of CUR and CUR/DOX micelles on Doxorubicin-resistant ovarian carcinoma cell lines, A2780/Adr after 4-44 hours of contact has been determined. The columns show the cell viability at CUR/DOX (uM) concentrations 40/0.8, 20/0.4, 10/0.2, 5/0.1, 2.5/0.05. and 1.25/0.025. Experimental data comparing the cell viability of CUR Micelles, CUR-G1_1 Micelles, CUR-G1_{—2} Micelles, CUR/DOX Micelles, CUR/DOX-G1_1 Micelles, and CUR/DOX-G1_2 Micelles is shown.

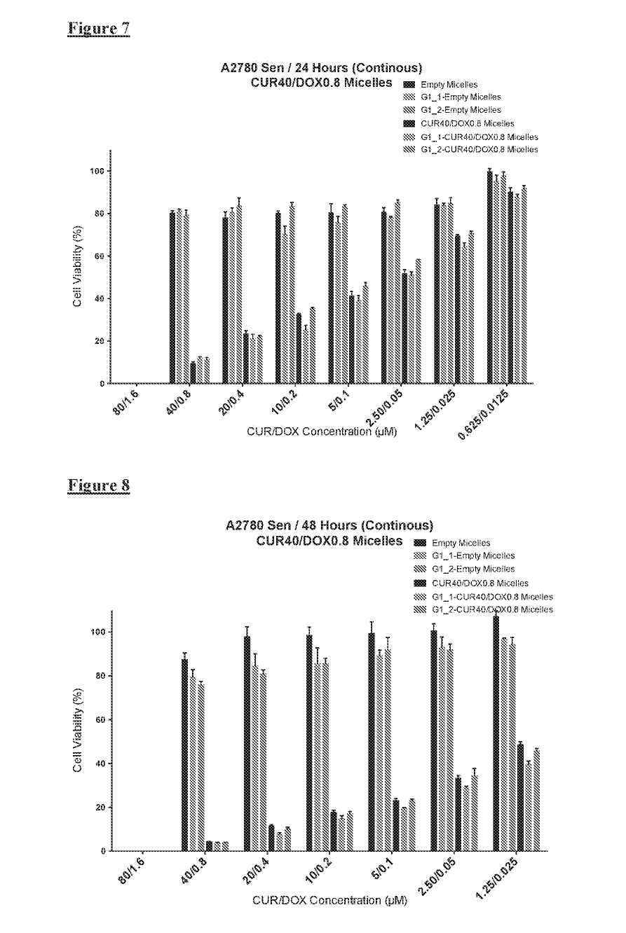

As illustrated in FIG. 7, the efficacy of CUR40/DOX0.8 micelles on Doxorubicin-sensitive ovarian carcinoma cell lines, A2780/Sen after 24 hours of continuous contact has been determined. The columns show the cell viability at CUR/DOX (uM) concentrations 80/1.6, 40/0.8, 20/0.4, 10/0.2, 5/0.1, 2.5/0.05, 1.25/0.025, and 0.625/0.0125. Experimental data comparing the cell viability of Empty Micelles, G1_1-Empty Micelles, G1_2-Empty Micelles, CUR40/DOX0.8 Micelles, G1_1-CUR40/DX0.8 Micelles, and G1_2-CUR40/DOX0.8 Micelles is shown.

As illustrated in FIG. 8, the efficacy of CUR40/DOX0.8 micelles on Doxorubicin-sensitive ovarian carcinoma cell lines, A2780/Sen, after 48 hours of continuous contact has been determined. The columns show the cell viability at CUR/DOX (uM) concentrations 80/1.6, 40/0.8, 20/0.4, 10/0.2, 5/0.1, 2.5/0.05, and 1.25/0.025. Experimental data comparing the cell viability of Empty Micelles, G1_1-Empty Micelles, G1_2-Empty CUR40/DOX0.8 Micelles, G1_1-CUR40/DOX0.8 Micelles, and G1_2-CUR40/DOX0.8 Micelles is shown.

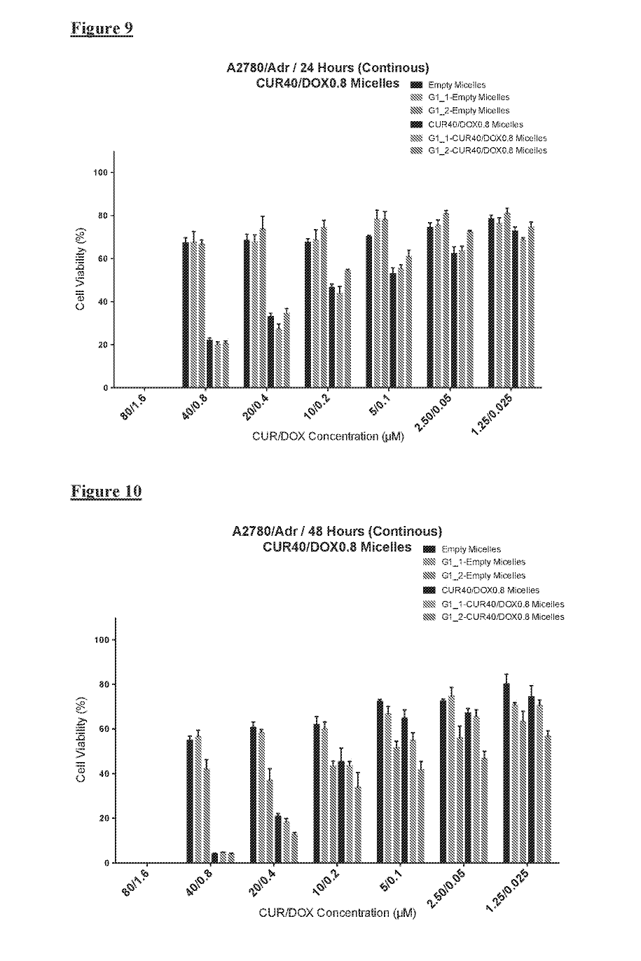

As illustrated in FIG. 9, the efficacy of CUR40/DOX0.8 micelles on Doxorubicin-resistant ovarian carcinoma cell lines, A2780/Adr after 24 hours of continuous contact has been determined. The columns show the cell viability at CUR/DOX (uM) concentrations 80/1.6, 40/0.8, 20/0.4, 10/0.2, 5/0.1, 2.5/0.05, and 1.25/0.025. Experimental data comparing the cell viability of Empty Micelles, G1_1-Empty Micelles, G1_2-Empty CUR40/DOX0.8 Micelles, G1_1-CUR40/DOX0.8 Micelles, and G1_2-CUR40/DOX0.8 Micelles is shown.

As illustrated in FIG. 10, the efficacy of CUR40/DOX0,8 micelles on Doxorubicin resistant ovarian carcinoma cell lines, A2780/Adr, after 48 hours of continuous contact has been determined. The columns show the cell viability at CUR/DOX (uM) concentrations 80/1.6, 40/0.8, 20/0.4, 10/0.2, 5/0.1, 2.5/0.05, and 1.25/0.025. Experimental data comparing the cell viability of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles is shown.

### Example 7

### Efficacy on Colon Cancer Cell Lines

In one embodiment, the inventor has demonstrated the efficacy of the compounds and micelles disclosed herein on human colon carcinoma cell line HCT-116.

As illustrated in FIG. 11, the efficacy of free CUR, free DOX, and free CUR/DOX on HCT-116 cells after 4-44 hours of contact has been determined. The columns illustrate the cell viability (%) at CUR/DOX (uM) concentrations: 40/0.8, 20/0.4, 10/0.2, 5/0.1, 2.5/0.05, and 1.25/0.025.

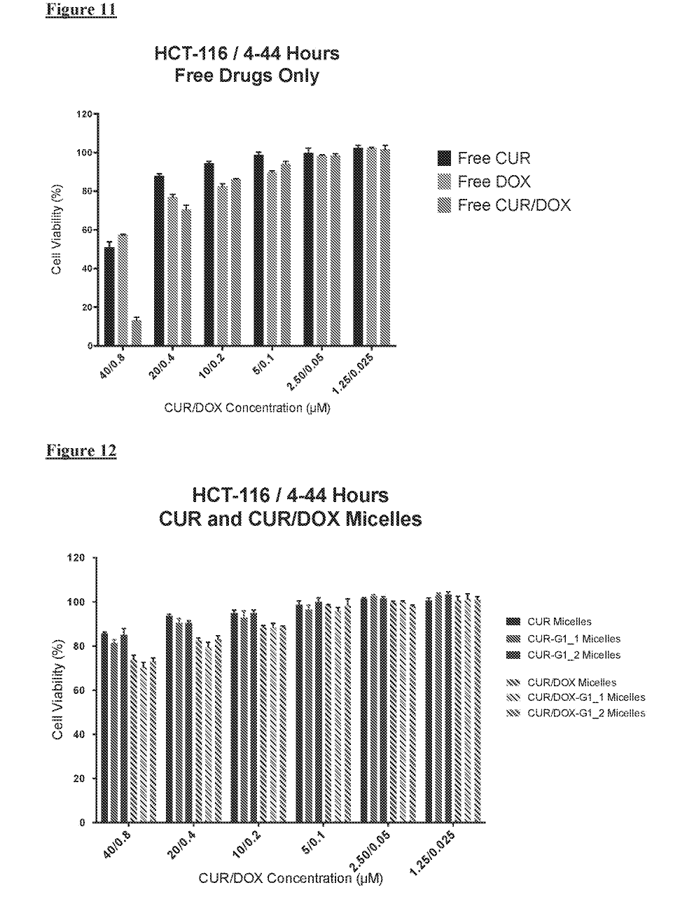

As illustrated in FIG. 12, the efficacy of CUR and CUR/DOX micelles on HCT-116 cells after 4-44 hours of contact has been determined. The columns show the cell viability at CUR/DOX (uM) concentrations 40/0.8, 20/0.4, 10/0.2, 5/0.1, 2.5/0.05, and 1.25/0.025. Experimental data comparing the cell viability of CUR Micelles, CUR-G1_1 Micelles, CUR-G1_2 Micelles, CUR/DOX Micelles, CUR/DOX-G1_1 Micelles, and CUR/DUX-G1_2 Micelles is shown.

As illustrated in FIG. 13, the efficacy of CUR40/DOX0.8 micelles HCT-116 cells after 24 hours of continuous contact has been determined. The columns show the cell viability at CUR/DOX (uM) concentrations 80/1.6, 40/0.8, 20/0.4, 10/0.2, 5/0.1, 2.5/0.05, and 1.25/0.025. Experimental data comparing the cell viability (%) of Empty Micelles, G1_1-Empty Micelles, G1_2-Empty Micelles, CUR40/DOX0.8 Micelles, G1_1-CUR40/DOX0.8 Micelles, and G1_2-CUR40/DOX0.8 Micelles is shown.

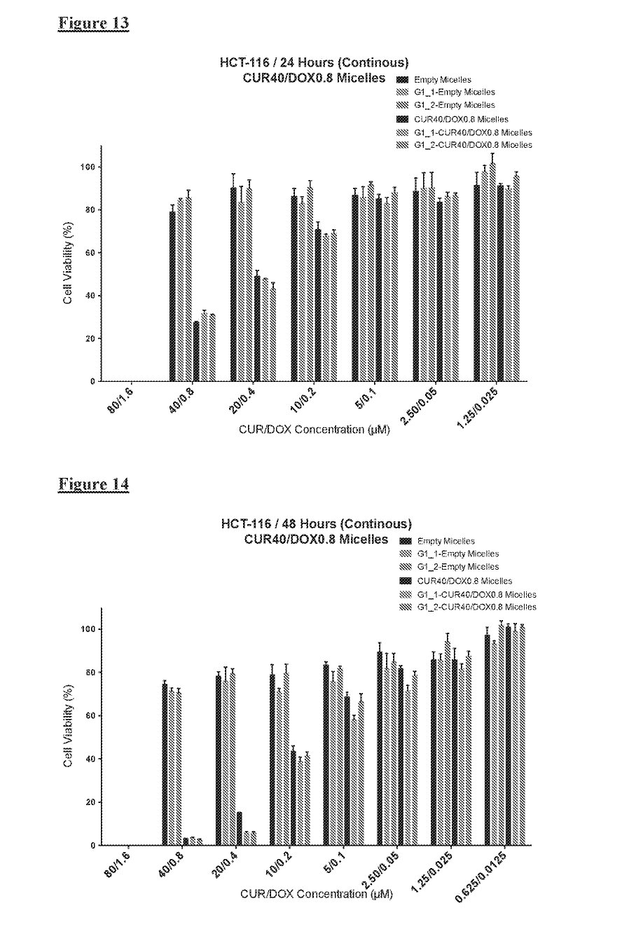

As illustrated in FIG. 14, the efficacy of CUR40/DOX0.8 micelles on HCT-116 cells after 48 hours of continuous contact has been determined. The columns show the cell viability at CUR/DOX (uM) concentrations 80/1.6, 40/0.8, 20/0.4, 10/0.2, 5/0,1, 2.5/0.05, and 1.25/0.025. Experimental data comparing the cell viability (%) of Empty MiCelles, G1_1-Empty Micelles, G1_2-Empty CUR40/DOX0.8 Micelles, G1_1-CUR40/DOX0.8 Micelles, and G1_2-CUR40/DOX0.8 Micelles is shown.

### Example 8

### Efficacy on Brain Cancer Cell Line

In one embodiment, the efficacy of the compounds and micelles disclosed herein has been demonstrated on human primary glioblastoma cell line U87MG. GLUT 1_1 and GLUT 1_2 scFv are two anti-Glut1 scFv's that was produced. The experiments were performed with U87MG monolayer treated with micelles. So far, this cell line was more responsive line to GLUT 1_1 and GLUT 1_2 scFv. 48 hours of contact was too much for this cell line because the cytotoxicity effect was saturated with drugs. However for 24 hours, the row and column effects were significant means that there was a dose response and also the antibody targeting effect. Significant difference corresponded to difference between non-targeted micelles to GLUT 1_1 or GLUT 1_2 targeted micelles, two-way ANOVA, P<0.05. In accordance with various embodiments, administration to U87MGs responded well to GLUT 1_2.

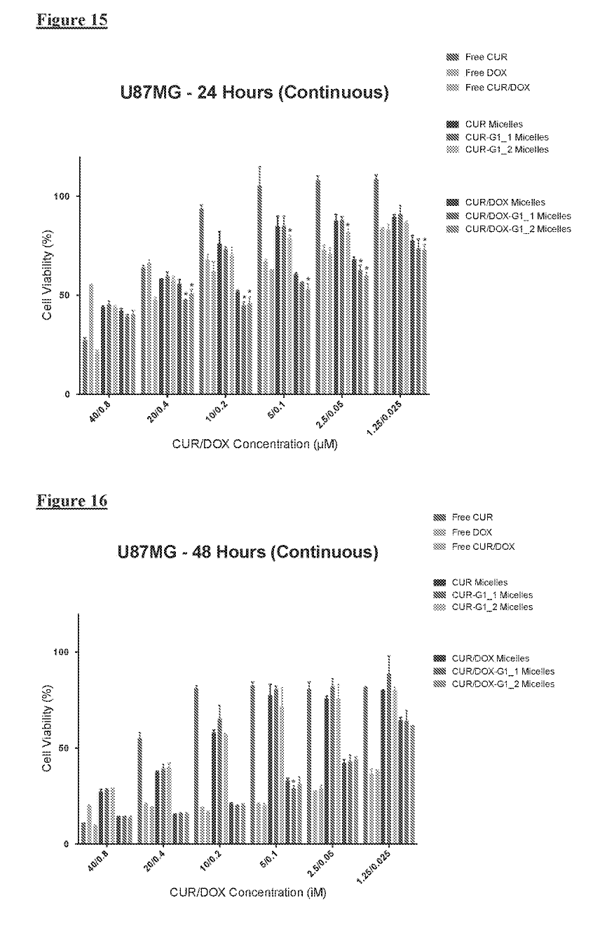

As illustrated in FIG. 15, the efficacy of free drugs and micelles on U87MG cells after 24 hours of continuous contact has been determined. The columns show the cell viability at CUR/DOX (uM) concentrations 40/0.8, 20/0.4, 10/0.2, 5/0.1, 2.5/0.05, and 1.25/0.025. Experimental data comparing the cell viability (%) of free CUR, free DOX, free CUR/DOX, CUR Micelles, CUR-G1_1 Micelles, CUR-G1_2 Micelles, CUR/DOX Micelles, CUR/DOX-G1_1 Micelles, and CUR/DOX-G1_2 Micelles is shown.

As illustrated in FIG. 16, the efficacy of free drugs and micelles on U87MG cells after 48 hours of continuous contact has been determined. The columns show the cell viability (%) at CUR/DOX (uM) concentrations 40/0.8, 20/0.4, 10/0.2, 5/0.1, 2.5/0.05, and 1.25/0.025. Experimental data comparing the cell viability of free CUR, free DOX, free CUR/DOX, CUR Micelles, CUR-G1_1 Micelles, CUR-G1_2 Micelles, CUR/DOX Micelles, CUR/DOX-G1_1 Micelles, and CUR/DOX-G1_2 Micelles is shown.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in sonic instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed, herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including, the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A composition comprising:
a. a micelle construct;
b. an antibody single chain fragment variable (scFv);
c. a NF-kb inhibitor; and
d. a topoisomerase II inhibitor.

2. The composition of claim 1, wherein the scFv is conjugated to the micelle construct.

3. The composition of claim 1, wherein the micelle construct s conjugated to the NF-kb inhibitor.

4. The composition of claim 1, wherein the micelle construct is conjugated to the NF-kb inhibitor and the topoisomerase II inhibitor.

5. The composition of claim 1, wherein the NF-kb inhibitor is curcumin or a pharmaceutical equivalent, analog, derivative, and/or salt thereof.

6. The composition of claim 1, wherein the topoisomerase II inhibitor is dox or a pharmaceutical equivalent, analog, derivative, and/or salt thereof.

7. The composition of claim 1, wherein the scFv is a glut-1_1 or glut-1_2 scFv.

8. The composition of claim 1, wherein the micelle construct is targeted to bind to glut-1_1 and/or glut-1_2 scFv.

9. The composition of claim 1, wherein the composition forms a targeted micelle.

10. The composition of claim 9, wherein the molecular size of the targeted mice is 30 nm or less.

11. The composition of claim 1, further comprising a pharmaceutically acceptable carrier.

12. A method of delivering a drug molecule to a tumor site of a subject comprising:
a. attaching the drug molecule to a targeted micelle, wherein the targeted micelle comprises a micelle construct and an antibody single chain fragment variable (scFv); and
b. delivering the drug molecule attached to the targeted micelle to the tumor site through intracellular delivery.

13. The method of claim 12, wherein the drug molecule comprises dox and/or curcumin or a pharmaceutical equivalent, analog, derivative, and/or salt thereof.

14. The method of claim 12, wherein the molecular size of the targeted micelle is 30 nm or less.

15. The method of claim 12, wherein the tumor is a doxorubicin-resistant tumor.

16. The method of claim 12, wherein the subject is human.

17. A method of treating cancer comprising administering to a subject in need thereof, a therapeutically effective dosage of a composition comprising a micelle construct, an antibody single chain fragment variable (scFv), a NF-kb inhibitor, and a topoisomerase II inhibitor.

18. The method of treating cancer of claim 17, wherein the composition is administered to the subject intravenously.

19. The method of treating cancer of claim 17, wherein the subject is human.

20. The method of treating cancer of claim 17, wherein the cancer is brain cancer.

21. The method of treating cancer of claim 17, wherein the cancer is ovarian cancer.

22. The method of treating cancer of claim 17, wherein the cancer is colon cancer.

23. The method of treating cancer of claim 17, wherein the cancer is a doxorubicin-resistant cancer.

24. The method of treating cancer of claim 17, wherein the molecular size of the micelle is 30 nm or less.

25. A method of inhibiting cell growth of a tumor cell, comprising:
a. providing a composition comprising a micelle construct, an antibody single chain fragment variable (scFv), a NF-kb inhibitor and a topoisomerase II inhibitor; and
b. inhibiting cell growth by administering a therapeutically effective dosage of the composition to the tumor cell.

26. The method of claim 25, wherein the topoisomerase inhibitor comprises Dox.

27. The method of claim 25, wherein the NF-kB inhibitor comprises curcumin.

28. The method of claim 25, wherein the tumor cell is a brain cancer cell, a ovarian cancer cell, and/or colon cancer cell.

29. A nanoconjugate, comprising:
a. a micelle construct;
b. an antibody single chain fragment variable (scFv);
c. a NT-kb inhibitor; and
d. a topoisomerase II inhibitor; and
wherein the micelle construct, the antibody single chain fragment variable (scFv), the NF-kb inhibitor, and the topoisomerase II inhibitor are conjugated to one another.

30. The nanoconjugate of claim 29, wherein the nanoconjugate has a molecular size between 20 nm and 50 nm.

31. The nanoconjugate of claim 29, wherein the nanoconjugate has a molecular size between 10 nm and 20 nm.

32. The nanoconjugate of claim 29, wherein the nanoconjugate has a molecular size less than 20 nm.

33. The nanoconjugate of claim 29, wherein the nanoconjugate is enclosed by a micelle.
